(19) United States
(12) Patent Application Publication
(10) Pub. No.: US 2024/0108225 A1
(43) Pub. Date: Apr. 4, 2024

(54) MULTI-WAVELENGTH TIME-RESOLVED LASER SPECKLE CONTRAST IMAGING (MTR-LSCI) OF TISSUE HEMODYNAMICS AND METABOLISM

(71) Applicant: University of Kentucky Research Foundation, Lexington, KY (US)

(72) Inventors: Guoqiang Yu, Lexington, KY (US); Faraneh Fathi, Lexington, KY (US); Chong Huang, Lexington, KY (US); Siavash Mazdeyasna, Riverdale Park, MD (US); Mingjun Zhao, New York, NY (US); Lei Chen, Lexington, KY (US)

(21) Appl. No.: 18/473,051

(22) Filed: Sep. 22, 2023

Related U.S. Application Data
(60) Provisional application No. 63/408,921, filed on Sep. 22, 2022.

(51) Int. Cl. A61B 5/00; A61B 5/026

(52) U.S. Cl. CPC A61B 5/0075; A61B 5/0261; A61B 5/4848; A61B 2503/40

(57) ABSTRACT

A noncontact, multi-wavelength time-resolved laser speckle contrast imaging (MTR-LSCI) technique provides for continuous, fast and high-resolution 2D mapping of tissue blood flow (BF) and tissue blood oxygen saturation (StO₂) at different depths of target tissue. MTR-LSCI illuminates the tissue with picosecond-pulsed, coherent, widefield light at least at two different wavelengths in the near-infrared range (600-1100 nm) and synchronizes a high-resolution, gated single-photon avalanche diode (SPAD) camera to capture BF and StO₂ maps at different depths of target tissue, wherein the imaging depth depends on light propagation inside a tissue volume, captured by the time-gated camera. The reconstruction of BF and StO₂ maps can be dramatically expedited by incorporating highly parallelized computation and convolution functions. The performance of MTR-LSCI was evaluated using head-simulating phantoms with known properties and in-vivo rodents with varied hemodynamic challenges to the brain.

### CROSS REFERENCE TO RELATED APPLICATIONS

This application claims the benefit of U.S. provisional patent application No. 63/408,921, filed Sep. 22, 2022, the contents of which are herein incorporated by reference.

### BACKGROUND OF THE INVENTION

### 1. Field of the Invention

Embodiments of the invention relate generally to apparatus and methods for determining tissue hemodynamics and metabolism. More particularly, embodiments of the invention relate to a noncontact, multi-wavelength time-resolved laser speckle contrast imaging (MTR-LSCI) apparatus and method of using the apparatus to determine tissue hemodynamics in a subject.

### 2. Description of Prior Art and Related Information

The following background information may present examples of specific aspects of the prior art (e.g., without limitation, approaches, facts, or common wisdom) that, while expected to be helpful to further educate the reader as to additional aspects of the prior art, is not to be construed as limiting the present invention, or any embodiments thereof, to anything stated or implied therein or inferred thereupon.

Blood flow (BF) carries nutrition and oxygen to tissues while also removing metabolic waste products. When oxygen demand exceeds oxygen supply, organ/tissue malfunction or permanent tissue damage may occur depending on the severity and duration of oxygen deprivation. Because BF and tissue blood oxygen saturation (StO₂) may not be coupled, combination of BF and STO₂ measurements enables distinguishing causes of tissue ischemia and tissue hypoxia occurred in many diseases including cerebral disease, cardiovascular disease, peripheral vascular disease, cancer, diabetes, burn/wound injury, angiogenesis or tissue/vascular reconstruction injury. From BF and STO₂ measurement results, one can derive the metabolic rate of tissue oxygen consumption (TMRO2), another important functional parameter tightly associated with tissue pathophysiology. Taken together, simultaneous measurements of BF and StO₂ enable the diagnosis of various diseases and the monitoring of related medical interventions.

Currently available neuroimaging technologies for deep tissue hemodynamics/metabolism measurements include magnetic resonance imaging (MRI), positron emission tomography (PET), x-ray computed tomography (CT) and transcranial doppler ultrasound (TCD). While useful, these imaging modalities present a host of issues that restrict their applications. MRI, PET, and CT are large, expensive, and difficult to use for continuous and longitudinal monitoring of tissue hemodynamics. PET uses ionizing radiation and CT involves exposing the patient to a significant amount of radiation, thus limiting their clinical applications. TCD enables the detection of blood flow in large vessels but is not sensitive to microcirculation.

By contrast, optical imaging instruments are portable, inexpensive, and fast, enabling continuous assessment of tissue hemodynamics in the microvasculature at the bedside. Laser speckle contrast imaging (LSCI) and optical intrinsic signal imaging (OISI) with widefield illumination and charge-coupled device/complementary-metal-oxide-semiconductor (CCD/CMOS) camera detection enable fast and high-resolution 2D mapping of cerebral blood flow (CBF) and cerebral blood oxygen saturation (ScO₂) on cortices of rodents, respectively. However, due to their limited penetration depth (<1 mm), LSCI and OISI require the retraction of the animal's scalp and/or an invasive cranial window for cortical imaging. As a result, LSCI and OISI are difficult to use for noninvasive deep brain imaging.

Conventional diffuse optical technologies such as near-infrared spectroscopy (NIRS) and diffuse correlation spectroscopy (DCS) use continuous-wave near-infrared (NIR) point sources and discrete photodetectors to noninvasively measure ScO₂ and CBF respectively in deep brains (up to centimeters). Correspondingly, NIR diffuse optical tomography (DOT) and diffuse correlation tomography (DCT) use dense arrays of sources and detectors to yield boundary measurements across numerous source-detector (S-D) pairs and solve inverse problems to reconstruct 3D images of ScO₂ and CBF, respectively. Tomographic 3D reconstructions by these methods need offline solving of ill-posed nonlinear inverse problems, which are complex and very time consuming. More recently, time-resolved NIRS (TR-NIRS) and time-resolved DCS (TR-DCS) use pulsed point sources and discrete single-photon avalanche diodes to measure temporal point-spread functions and autocorrelation functions for quantifying ScO₂ and CBF in deep brains, respectively. However, most systems suffer from limited numbers of discrete sources and photodetectors, thus taking sparse S-D pair measurements on a limited region-of-interest (ROI) of the head. Expanding the ROI to cover a significant portion of the head introduces great challenges in high-channel-count instrumentation, imaging array ergonomics and data quality management. As a result, their spatial resolutions and head coverages (ROIs) are limited, thus inhibiting the localization of regional brain activations. Moreover, collected cerebral signals are inherently influenced by partial volume artifacts from overlayer tissues of scalp and skull.

As can be seen, there is a need for systems and methods for a noninvasive, affordable, and portable optical imaging device with the ability to track blood flow, oxygen levels and TMRO₂ of subjects.

### SUMMARY OF THE INVENTION

Embodiments of the present invention relate to an affordable and portable optical imaging device that is much smaller and less costly than MRI and CT imaging. Embodiments of the device, as disclosed herein, are noninvasive with the ability to track blood flow, oxygen levels, metabolism and cell health in tissue of subjects. Studies described herein are useful for establishing efficacy of the disclosed optical imaging device. Aspects described herein provide a unique, portable optical imaging system, which can be applied, for example, in neonatal intensive care units for continuous monitoring and instant management of brain injury and treatment in human newborns.

To address many of the deficiencies in competing imaging technologies, a novel, noncontact, affordable, multi-wavelength time-resolved laser speckle contrast imaging (MTR-LSCI) technique was developed that enables continuous, fast, and high-resolution 2D mapping of BF, StO₂, and TMRO₂ at different depths of target tissue. In contrast to other NIR technologies using point-source illuminations and discrete detectors, MTR-LSCI illuminates picosecond-pulsed, coherent, widefield NIR light onto the tissue and synchronizes a time-gated, single-photon avalanche diode (SPAD) camera to rapidly image BF, StO₂, and TMRO₂ distributions at different depths of target tissue. For example, by applying the time-gated strategy to synchronize the pulsed widefield illumination and camera opening time (i.e., time-resolved method), MTR-LSCI differentiates short and long photon paths through layered head tissues (i.e., scalp, skull, and brain) to precisely map CBF and ScO₂ distributions in subjects with different head scales (i.e., multiscale). Importantly, MTR-LSCI eliminates the need for time-consuming complex tomographic reconstruction of 3D images of ScO₂/CBF in conventional DOT/DCT technologies, thereby offering depth-sensitive 2D maps of ScO₂/CBF in near real time.

As described in greater detail below, a benchtop prototype was prepared and proof-of-concept studies were carried out to introduce the innovative MTR-LSCI as a photon pathlength resolved technology for imaging of CBF variations at different depths of the head. Head/flow-simulating phantoms with different thicknesses of top layers were created and imaged to evaluate depth sensitivity of MTR-LSCI technology. In vivo adult rodents were then imaged by the MTR-LSCI during CO₂ inhalations (increasing CBF globally) and during unilateral and bilateral transient ligations of carotid arteries (reducing CBF regionally and globally). Results verified the capability of MTR-LSCI for continuous, fast, and high-density, 2D mapping CBF changes at different depths.

In some aspects of the present invention, a device for measuring hemodynamics comprises at least two nanosecond or picosecond pulsed near infrared laser diodes; a time-gated camera; an image sensor with a resolution of at least 512×512 photon-counting pixels; and a gating mechanism.

In some aspects of the present invention, a method of measuring cerebral hemodynamics in a subject comprises (a) positioning a device for measuring hemodynamics, as described herein, in proximity to, but not directly touching, a subject; (b) using the pulsed near infrared laser diodes of the device to apply pulsed widefield illumination at multiple wavelengths; (c) using the gating mechanism to apply a time-gated function with nanosecond gate width; (d) setting the two nanosecond or picosecond pulsed near infrared laser diodes to different wavelengths and (e) measuring tissue blood flow, concentration of oxy-hemoglobin (HbO₂), and concentration of deoxy-hemoglobin (Hb).

In some aspects of the present invention, a device for measuring cerebral hemodynamics comprises at least two nanosecond or picosecond pulsed near infrared laser diodes; a time-gated camera with a resolution of at least 512×512 photon-counting pixels; and a gating mechanism.

In some aspects of the present invention, a method of measuring hemodynamics in a subject involves (a) positioning a device, such as the device for measuring cerebral hemodynamics, as described in the previous paragraph and as further described here within, in proximity to, but not directly touching, a subject; (b) using the pulsed near infrared lasers of the device to apply pulsed widefield illumination at multiple wavelengths; (c) using the gating mechanism to apply a time-gated function in with nanosecond gate width; (d) setting the at least two nanosecond or picosecond pulsed near infrared lasers to different wavelengths; and (e) measuring tissue blood flow, concentration of HbO₂, and concentration of Hb.

In some embodiments of the presently-disclosed subject matter, the wavelengths are set to 785 nm and 830 nm. In some embodiments, the gating mechanism is synchronized with the pulsed near infrared lasers using multiple successive overlapping gates. In further embodiments, the methods described herein above further comprises calculating the cerebral metabolic rate of oxygen consumption (CMRO₂). In some embodiments, the device is positioned in proximity to the subject's head. In further embodiments, the device is positioned in proximity to the subject's skin. In further embodiments, the subject is a human. In other embodiments, the subject is a neonate. In further embodiments, the neonate is diagnosed with HIE. In other embodiments, the neonate is diagnosed with peripheral vascular disease, burn/wound injury, angiogenesis, or tissue/vascular reconstruction injury.

Embodiments of the present invention provide a system for noncontact, multiwavelength, time-resolved laser speckle contrast imaging (MTR-LSCI) of tissue blood flow, tissue blood oxygenation, and metabolic rate of tissue oxygen consumption in a subject, comprising at least two pulsed laser sources, each capable of emitting light pulses in nanosecond or picosecond width at near-infrared (NIR) range of 600-1100 nm, for illuminating tissue; at least one diffuser in front of each of the at least two pulsed laser sources to generate a wide-field illumination; a time-gated camera; a controller to synchronize the time-gated camera and the at least two pulsed laser sources at 10-80 MHz for data collection; and a computing device having a processor for processing data to generate hemodynamic images on a display.

In some embodiments, which may be combined with the above embodiment, the system further includes algorithms incorporating parallel computation and convolution functions to process received images and generate the hemodynamic images to the display.

In some embodiments, which may be combined with the above embodiments, the time-gated camera has a gate step resolution of picoseconds.

In some embodiments, which may be combined with the above embodiments, the time-gated camera has a gate width of nanoseconds.

In some embodiments, which may be combined with the above embodiments, the time-gated camera has a spatial resolution of at least 256×512 single-photon-counting pixels.

In some embodiments, which may be combined with the above embodiments, the system further includes at least one filter within the time-gated camera path to minimize an impact of ambient light on a detection NIR spectra.

In some embodiments, which may be combined with the above embodiments, the system further includes at least two polarizers across each of the at least two pulsed laser sources and the time-gated camera path to reduce an influence of source reflections directly from a tissue surface.

In some embodiments, which may be combined with the above embodiments, the system further includes at least one zoom lens attached to the time-gated camera to adjust the region-of-interest (ROI)/field-of-view (FOV).

In some embodiments, which may be combined with the above embodiments, the subject is one of a human or an animal.

Embodiments of the present invention further provide a method of measuring tissue blood flow, tissue blood oxygenation, and metabolic rate of tissue oxygen consumption at different depths in a subject, comprising positioning a multi-wavelength, time-resolved laser speckle contrast imaging (MTR-LSCI) device in proximity to the subject, the MTR-LSCI device including at least two pulsed laser sources, each capable of emitting light pulses in nanosecond or picosecond width at near-infrared (NIR) range of 600-1100 nm, for illuminating tissue, at least one diffuser in front of each of the at least two pulsed laser sources to generate a wide-field illumination, at least one optical switch to switch between the at least two pulsed light sources, a time-gated camera, at least one zoom lens attached to the time-gated camera to adjust the ROI/FOV, a controller to synchronize the time-gated camera and the at least two pulsed laser sources at 10-80 MHz for data collection, and a computing device having a processor for processing data to generate hemodynamic and metabolic images to a display; using the at least two pulsed laser sources to apply pulsed widefield illumination at multiple wavelengths; setting the at least two pulsed laser sources to different wavelengths; and measuring at least one of tissue blood flow, tissue blood oxygenation, and metabolic rate of tissue oxygen consumption in the subject.

In some embodiments, which may be combined with any of the above embodiments, the method maps blood flow (BF) distributions at different depths of target tissue, wherein the imaging depth depends on light propagation inside a tissue volume, captured by the time-gated camera.

In some embodiments, which may be combined with any of the above embodiments, the method maps blood flow (BF) distributions at different depths of target tissue, wherein the BF is calculated based on detected diffuse laser speckle fluctuations resulting from motions of red blood cells in a target tissue volume.

In some embodiments, which may be combined with any of the above embodiments, the method maps parameters of oxy-hemoglobin concentration ([HbO2]), deoxy-hemoglobin concentration ([Hb]), and tissue blood oxygen saturation (StO2) at different depths of target tissue, wherein the parameters are calculated based on detected light intensity attenuations by a target tissue volume.

In some embodiments, which may be combined with any of the above embodiments, the method determines a TMRO2, wherein the TMRO2 is calculated based on a measured BF and StO2.

In some embodiments, which may be combined with any of the above embodiments, the tissue is any part of a body of the subject.

In some embodiments, which may be combined with any of the above embodiments, the subject is either healthy or is diagnosed with a disease associated with altered tissue blood flow and oxygenation, cerebral disease, cardiovascular disease, peripheral vascular disease, cancer, diabetes, burn/wound injury, angiogenesis or tissue/vascular reconstruction injury.

In some embodiments, which may be combined with any of the above embodiments, the subject is under medical interventions.

In some embodiments, which may be combined with any of the above embodiments, wherein the method further includes determining an efficacy of interventions.

Embodiments of the present invention further provide an integrated instrument for performing MTR-LSCI measurements, comprising at least two nanosecond or picosecond pulsed NIR lasers, coupled with diffusers to deliver wide-field illumination to a target tissue volume; a time-gated, single-photon avalanche diode (SPAD) camera to capture intensity images and deliver the intensity images to a computer processor, wherein the computer processor includes control software to synchronize the at least two pulsed lasers and the time-gated SPAD camera for data collection; and a computing device to process received images using parallel computation and convolution functions and generate hemodynamic and metabolic images on a display.

In some embodiments, which may be combined with any of the above embodiments, the MTR-LSCI instrument is in proximity to, but not directly touching, the subject.

In some embodiments, which may be combined with any of the above embodiments, a microlens array is attached to the camera to enhance the detection sensitivity.

In some embodiments, which may be combined with any of the above embodiments, wavelengths of the at least two pulsed lasers are set to 785 nm and 830 nm.

These and other features, aspects and advantages of the present invention will become better understood with reference to the following drawings, description and claims.

### BRIEF DESCRIPTION OF THE DRAWINGS

Some embodiments of the present invention are illustrated as an example and are not limited by the figures of the accompanying drawings, in which like references may indicate similar elements.

FIG. 1 illustrates an experimental MTR-LSCI setup, according to an exemplary embodiment of the present invention;

FIG. 2A illustrates a conceptual representation of a single wavelength MTR-LSCI system to capture 2D flow maps at different depths using different gates, for clarity, only seven gate positions with the interval time of 10 ns (10×1 ns delay time) are illustrated, with the selected offset time of 30.5 ns, shallow and deep depth images were captured at gate positions 4 and 5, respectively;

FIG. 2B illustrates a gate camera intensity profile detected from a calibration tissue phantom at different depths (gates), where the preset parameters include laser frequency: 20 MHz (50 ns); offset time: 0 ns; gate delay time: 1 ns; number of gates: 200; detection time window: 200 ns=200×1 ns, and generally, the offset time ranging from 30 ns to 32 ns was chosen from the shaded region of FIG. 2A, between positions 4 and 5, where longer offset time corresponds deeper imaging depth;

FIG. 2C illustrates a gate camera intensity profile detected from experimental tissue phantoms and animals at different depths (gates) using preset parameters including laser frequency: 20 MHz (50 ns); offset time: 30.5 ns; gate delay time: 18 ps; number of gates: 200; detection time window: 3.6 ns=200×18 ps;

FIG. 3A illustrates an exemplary MTR-LSCI setup and for capturing tissue hemodynamics and metabolism maps at different depths (M1 and M2);

FIG. 3B illustrates an exemplary representation of the principle of MTR-LSCI for capturing tissue hemodynamics and metabolism maps at different depths (M1 and M2) using different time gates;

FIG. 4 illustrates a flowchart for processing single-wavelength MTR-LSCI data and generating a 2D BFI map, where two nested for-loops were used to carry out computational processes including the outer for-loop to iterate through all gated images (solid outer box) and inner parfor-loop to process time-course data in parallel (solid inner box), where steps inside the dashed red box were applied to every intensity gated image to eventually generate a 2D map of kₛ. m: number of gated intensity images; n: number of time-course intensity images; G_{m,n}: gated image taken at Gate #m and Time #n; Tₙ: time folder; w: pixel window size, I: raw intensity image, <I>: mean intensity image; <I>²: square of mean intensity image; <I²>: mean of squared intensity image; kₛ: laser speckle contrast map; k_{α}: average laser speckle contrast at Gate #m over all time folders (T₁:T_{N}); BFI: blood flow index;

FIG. 5 illustrates a sequential overview of MTR-LSCI data processing for generating BFI, StO₂ and TMRO₂ Maps, where the MTR-LSCI collects Kₛ(λ) and I_{λ} data with the at least two wavelengths (e.g., λ₁ & λ₂), which are used to derive multiple functional parameters;

FIG. 6 illustrates experimental results in head-simulating phantoms, where (a)-(c) show top, bottom, and 3D views of a 3D-printed UK logo solid phantom without flow (i.e., flow index=0) with varying top layer thicknesses (1, 2, 3 mm) the empty UK letter channels were filled in liquid phantom solution with Intralipid particle flow (i.e., flow index=1) to generate flow contrasts, where (d)-(g) show reconstructed 2D maps of particle flow contrasts in three phantoms with the top layer thicknesses of 1, 2 and 3 mm respectively, generated by the MTR-LSCI at gates #60, #80, #100, and #120 respectively, where the preset parameters for MTR-LSCI measurements include laser frequency: 20 MHz (50 ns); offset time: 30.5 ns; gate delay time: 18 ps; number of gates: 200; detection time window: 3.6 ns=200×18 ps, where one hundred (100) time-course images at each gate were taken and averaged to increase SNRs;

FIG. 7A illustrates a raw intensity image of mouse skull with its scalp retracted, where a zoom lens connected to the MTR-LSCI was adjusted to scan a ROI of 20×10 mm² on the mouse skull;

FIGS. 7B and 7C illustrate reconstructed blood flow index (BFI) maps with the gated mode at Gate #0 and the intensity mode, respectively, where the camera exposure time for the intensity mode was 5.12 ms, and where preset parameters for the gated mode include laser frequency: 20 MHz (50 ns); offset time: 30.5 ns; gate delay time: 18 ps; number of gates: 80; detection time window:1.4 ns=80×18 ps and four hundred time-course images were taken and averaged for both intensity and gated modes to increase SNRs;

FIG. 8A illustrates a raw intensity image of an illustrative rat skull with its scalp retracted;

FIG. 8B illustrates reconstructed BFI maps before, during, and after CO₂ inhalation, taken at Gate #35, where, during the baseline and CO₂ inhalation phases, around 70 time-course images were averaged to create BFI maps with enhanced SNRs, and where, similarly, approximately 140 images were averaged during the recovery phase;

FIG. 8C illustrates a corresponding time-course rCBF changes in the selected brain region in the same illustrative rat;

FIG. 8D illustrates an average time-course rCBF changes (mean value ±standard error) over 6 rats, where the shaded area represents standard errors;

FIG. 8E illustrates a comparison of average time-course changes in rCBF (mean values) at Gates #1, #10, #20, #35, and #50 over 6 rats, where, for clarity, only mean values at different gates (i.e., no standard errors) are presented, and where preset parameters for the gated mode include laser frequency: 20 MHz (50 ns); offset time: 30.5 ns; gate delay time: 18 ps; number of gates: 80; detection time window: 1.4 ns=200×18 ps;

FIG. 9A illustrates a raw intensity image of an illustrative rat skull with its scalp retracted;

FIG. 9B illustrates reconstructed BFI maps before, during, and after unilateral and bilateral CCA ligations and releases, followed by CO₂ euthanasia taken at Gate #35 in one illustrative rat, where, for most stages of ligations, ˜70 time-course images were averaged to enhance SNRs, while 28 time-course images were averaged during the bilateral ligation phase;

FIG. 9C illustrates a corresponding time-course rCBF changes at two hemispheres (RH and LH) in the same illustrative rat, measured continuously by TR-LSCI with a sampling rate of 0.2 Hz;

FIG. 9D illustrates an average time-course rCBF changes (mean value ±standard error) in each hemisphere over 4-6 rats, where the shaded area represents standard errors, and where the preset parameters for the gated mode include laser frequency: 20 MHz (50 ns); offset time: 30.5 ns; gate delay time: 18 ps; number of gates: 80; detection time window: 1.4 ns=200×18 ps; and

FIGS. 10A and 10B illustrate a table comparing features of optical neuroimaging technologies, including MTR-LSCI, according to embodiments of the present invention.

Unless otherwise indicated, the figures are not necessarily drawn to scale.

The invention and its various embodiments can now be better understood by turning to the following detailed description wherein illustrated embodiments are described. It is to be expressly understood that the illustrated embodiments are set forth as examples and not by way of limitations on the invention as ultimately defined in the claims.

### DETAILED DESCRIPTION OF THE PREFERRED EMBODIMENTS AND BEST MODE OF INVENTION

The terminology used herein is for the purpose of describing particular embodiments only and is not intended to be limiting of the invention. As used herein, the term “and/or” includes any and all combinations of one or more of the associated listed items. As used herein, the singular forms “a,” “an,” and “the” are intended to include the plural forms as well as the singular forms, unless the context clearly indicates otherwise. It will be further understood that the terms “comprises” and/or “comprising,” when used in this specification, specify the presence of stated features, steps, operations, elements, and/or components, but do not preclude the presence or addition of one or more other features, steps, operations, elements, components, and/or groups thereof.

Unless otherwise defined, all terms (including technical and scientific terms) used herein have the same meaning as commonly understood by one having ordinary skill in the art to which this invention belongs. It will be further understood that terms, such as those defined in commonly used dictionaries, should be interpreted as having a meaning that is consistent with their meaning in the context of the relevant art and the present disclosure and will not be interpreted in an idealized or overly formal sense unless expressly so defined herein.

In describing the invention, it will be understood that a number of techniques and steps are disclosed. Each of these has individual benefit and each can also be used in conjunction with one or more, or in some cases all, of the other disclosed techniques. Accordingly, for the sake of clarity, this description will refrain from repeating every possible combination of the individual steps in an unnecessary fashion. Nevertheless, the specification and claims should be read with the understanding that such combinations are entirely within the scope of the invention and the claims.

In the following description, for purposes of explanation, numerous specific details are set forth in order to provide a thorough understanding of the present invention. It will be evident, however, to one skilled in the art that the present invention may be practiced without these specific details.

The present disclosure is to be considered as an exemplification of the invention and is not intended to limit the invention to the specific embodiments illustrated by the figures or description below.

As is well known to those skilled in the art, many careful considerations and compromises typically must be made when designing for the optimal configuration of a commercial implementation of any system, and in particular, the embodiments of the present invention. A commercial implementation in accordance with the spirit and teachings of the present invention may be configured according to the needs of the particular application, whereby any aspect(s), feature(s), function(s), result(s), component(s), approach(es), or step(s) of the teachings related to any described embodiment of the present invention may be suitably omitted, included, adapted, mixed and matched, or improved and/or optimized by those skilled in the art, using their average skills and known techniques, to achieve the desired implementation that addresses the needs of the particular application.

Unless otherwise indicated, all numbers expressing quantities of ingredients, properties such as reaction conditions, and so forth used in the specification and claims are to be understood as being modified in all instances by the term “about”. Accordingly, unless indicated to the contrary, any numerical parameters set forth in this specification and claims are approximations that can vary depending upon the desired properties sought to be obtained by the presently-disclosed subject matter.

As used herein, the term “about,” when referring to a value or to an amount of mass, weight, time, volume, concentration or percentage is meant to encompass variations of in some embodiments ±20%, in some embodiments ±10%, in some embodiments ±5%, in some embodiments ±1%, in some embodiments ±0.5%, in some embodiments ±0.1%, in some embodiments ±0.01%, and in some embodiments ±0.001% from the specified amount, as such variations are appropriate to perform the disclosed method.

As used herein, ranges can be expressed as from “about” one particular value, and/or to “about” another particular value. It is also understood that there are a number of values disclosed herein, and that each value is also herein disclosed as “about” that particular value in addition to the value itself. For example, if the value “10” is disclosed, then “about 10” is also disclosed. It is also understood that each unit between two particular units are also disclosed. For example, if 10 and 15 are disclosed, then 11, 12, 13, and 14 are also disclosed.

As used herein, “optional” or “optionally” means that the subsequently described event or circumstance does or does not occur and that the description includes instances where said event or circumstance occurs and instances where it does not. For example, an optionally variant portion means that the portion is variant or non-variant.

As used herein, the term “subject” refers to a target of administration or medical procedure. The subject of the herein disclosed methods can be a human or animal. The subject may also be a mammal. Thus, the subject of the herein disclosed methods can be a human, nonhuman primate, horse, pig, rabbit, dog, sheep, goat, cow, cat, guinea pig or rodent. The term does not denote a particular age or sex. Thus, adult and newborn subjects, as well as fetuses, whether male or female, are intended to be covered. A “patient” refers to a subject afflicted with a disease or disorder. The term “patient” includes human and veterinary subjects.

As used herein, the term “diagnosed” means having been subjected to a physical examination by a person of skill, for example, a physician, and found to have a condition that can be diagnosed or treated by the compounds, compositions, or methods disclosed herein. For example, “diagnosed with hypoxic-ischemic encephalopathy” means having been subjected to a physical examination by a person of skill, for example, a physician, and found to have a condition that can be described as hypoxic-ischemic encephalopathy.

Broadly, embodiments of the present invention address many of deficiencies in existing neuroimaging technologies, such as poor spatial resolution, time-consuming reconstruction, low penetration depth, and contact-based measurement, with a novel, noncontact, multi-wavelength time-resolved laser speckle contrast imaging (MTR-LSCI) technique for continuous, fast and high-resolution 2D mapping of tissue blood flow (BF),tissue blood oxygen saturation (StO2), and the metabolic rate of tissue oxygen consumption (TMRO₂), at different depths of target tissue. MTR-LSCI illuminates the tissue with picosecond-pulsed, coherent, widefield light at least at two different wavelengths in the near-infrared range (600-1100 nm) and synchronizes a high-resolution, gated single-photon avalanche diode (SPAD) camera to capture BF,StO₂, and TMRO₂ maps at different depths of target tissue, wherein the imaging depth depends on light propagation inside a tissue volume, captured by the time-gated camera. The reconstruction of BF and StO₂ maps can be dramatically expedited by incorporating highly parallelized computation and convolution functions. The performance of MTR-LSCI was evaluated using head-simulating phantoms with known properties and in-vivo rodents with varied hemodynamic challenges to the brain. Results from these pilot studies demonstrated that MTR-LSCI enabled mapping cerebral hemodynamic variations at different depths with a sampling rate of up to 1 Hz and spatial resolutions ranging from tens of micrometers on the head surface to 1-2 millimeters in the deep brain. Further embodiments can provide a noncontact, fast, high-resolution, portable and affordable tissue hemodynamic imager for fundamental neuroscience research in animals and for translational studies in humans.

### MTR-LSCI Prototype

A benchtop single-wavelength MTR-LSCI device was developed for measuring tissue BF alone. A free space, picosecond-pulsed, single-mode laser worked as a coherence point source (wavelength: 775 nm; pulse width: 540 ps; spectral bandwidth: <1 nm; max power at 20 MHz: 2 Watt; Katana-08 HP, NKT Photonics). Two engineered optical diffusers (ED1-C20-MD and ED1-S20-MD, Thorlabs) were placed in front of the point source to generate the widefield illumination. The laser power was adjusted so that the maximum photon counts detected by the gated SPAD camera (SwissSPAD2, EPFL, Switzerland) were within its linearity range (<500 counts for 10-bit image). Correspondingly, the power of incident light from the MTR-LSCI on the tissue surface was less than 1.2 mW/cm2 (measured by a power meter), which is compliant with the Accessible Emission Limit Class 3R of the American National Standards Institute (ANSI) standard.

SwissSPAD2 is an ultra-high-speed single-photon camera, comprising 512×512 SPAD pixels that can be time-gated. In experiments, a resolution of 256×512 pixels was used to capture speckle contrast images at different depths. SwissSPAD2 achieves a combination of high sensitivity (50% photon detection probability at 520 nm), low pitch (16.38 Rm), moderate fill factor (10.5% fill factor equivalent to 31 μm SPAD active area radius), high temporal resolution (18 ps minimum gate shift), and low dark count rate (0.26 photon counts per second (cps)/μm2). Compared to the majority of other SPAD detectors, the combination of large spatial resolution (large number of pixels) and high temporal resolution (picosecond range) is the greatest advantage of the SwissSPAD2 camera.

A zoom lens (MLM3X-MP, Computar) was coupled to the camera for adjusting ROI size. The F/# of the zoom lens was set at 11 to satisfy the Nyquist criterion based on laser speckle and camera pixel sizes. A pair of polarizers (LPNIRE050-B and LPNIRE200-B, Thorlabs) were added crossing the source and detection paths to reduce the influence of source reflections directly from the tissue surface. A long-pass filter (>750 nm, FEL0750, Thorlabs) was used to minimize the impact of ambient light on MTR-LSCI measurements.

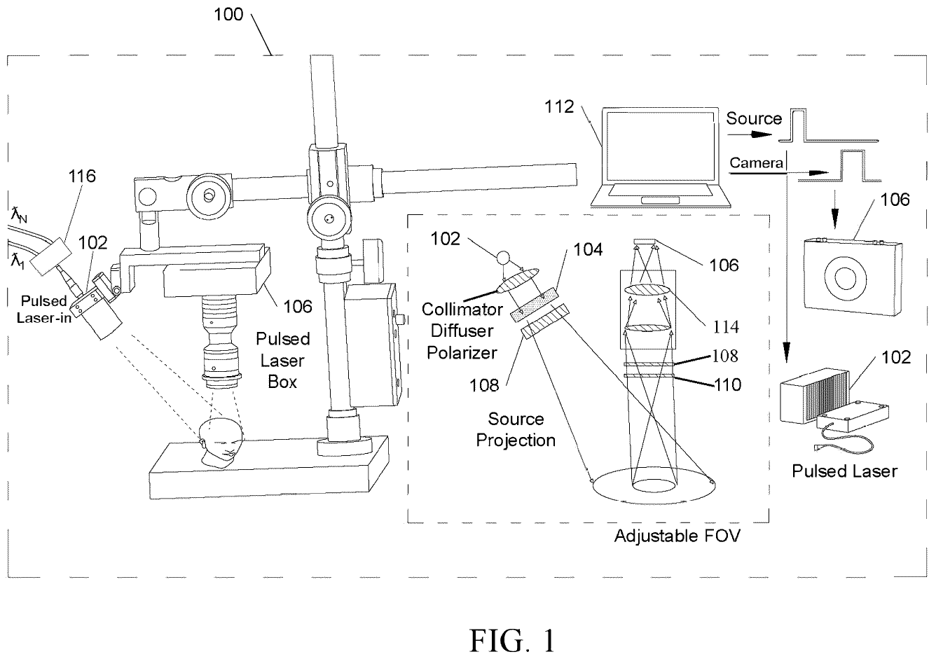

FIG. 1 shows a portable MTR-LSCI device 100 for measuring BF and StO₂ simultaneously. For illumination of target tissue, at least two pulsed laser sources 102 are turned on alternatively via an optical switch 116 or in free space, each capable of emitting light pulses in nanosecond or picosecond width at NIR range of 600-1100 nm (e.g., λ₁=785 nm and λ₂=830 nm). For detection, a customized SPAD512S camera 106 was used (Pi Imaging Technology) with integrated microlenses and a new operating system (C++) to improve imaging sensitivity, signal-to-noise ratio (SNR) and spatial-temporal resolution. These parts and others (collimator, diffusers 104, polarizers 108, filters 110 and zoom lens 114) can be mechanically integrated into a compact probe fixed on a 360° holder for easy alignment to a selected field of view (FOV)/ROI. Through electrical triggers in the range of 10-80 MHz, a computer 112, as a control unit, synchronizes the pulsed lasers 102 and SPAD512S camera 106 to automatically acquire MTR-LSCI data.

As described above, the portable MTR-LSCI system incorporates at least two nanosecond or picosecond pulsed lasers (λ≥2). The lasers are coupled with a time-gated camera, which can be conveniently housed within a compact probe fixed onto a 360-degree holder for easy alignment with the desired field FOV/ROI. A side illumination configuration can be achieved by positioning the lasers alongside the camera. The pulsed lasers can emit triggers within the range of 10-80 MHz to synchronize their operation with the time-gated camera. The noncontact MTR-LCSI system can be placed over any tissue/organ in animals and humans, enabling continuous imaging of BF and StO₂. Typical components of MTR-LSCI device include a minimum of two nanosecond or picosecond-pulsed lasers (λ≥2) to generate coherent point light, at least one optical diffuser to provide widefield illumination, at least a pair of polarizers to mitigate source reflections, a zoom lens for adjusting the FOV/ROI, a filter to minimize the influence of ambient light and a time-gated camera for capturing intensity images at varied depths of target tissue.

The operating system can be optimized to be “plug-and-play” so that a neuroscientist can easily use it without specialized training. The control panel of the computing device can have only a few buttons to control the device (e.g., start, marking events, stop), input boxes to set up parameters (e.g., ROI, camera pixel-window size, gated time, exposure time, and frame rate) and display windows to automatically report resulting images.

### MTR-LSCI Principle and Data Acquisition

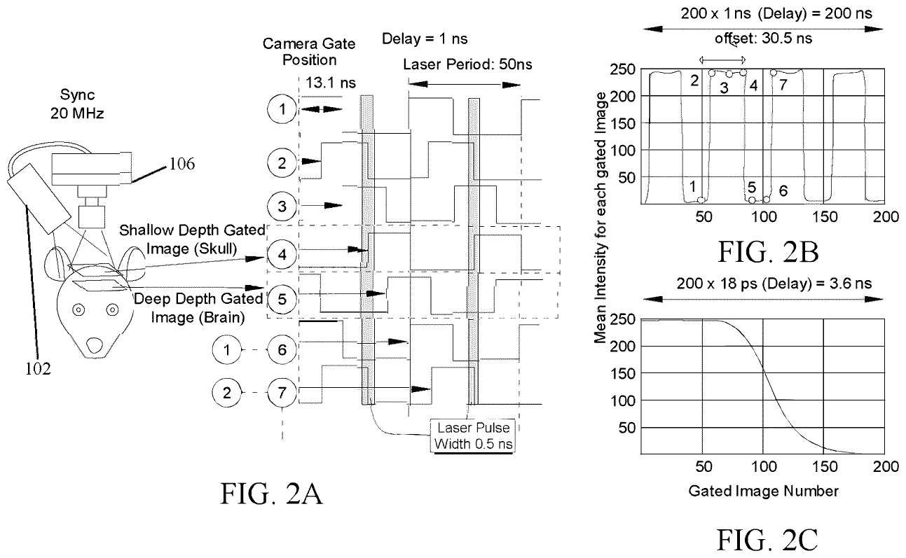

The SwissSPAD2 camera/sensor can operate with two modes: intensity mode and gated mode. The intensity mode, used as the conventional LSCI, operates by having the gate fully open during the exposure window and fully closed during the readout window. In the intensity mode, images are captured with an 8-bit depth, whereas the gated mode allows for capturing images with both 8-bit and 10-bit depths. FIGS. 2A through 2C show the principle and data acquisition of a single-wavelength MTR-LSCI with the gated mode. Time gating in SPAD cameras functions as a filter that only captures photons when they arrive within a specific time window. Through electrical triggers produced by the pulsed laser or a signal generator as the master control, the laser and SwissSPAD2 camera are synchronized at 20 MHz (equivalent period of 50 ns=1/20 MHz) for acquiring multiple gated images at different delay times (minimal 18 picoseconds). Overlapping gates are created when the delay time between adjacent gate windows (also known as gate step) is less than the actual camera gate width (13.1 ns used in this study). Using overlapping gates with a minimal gate delay time of 18 ps, sub-nanosecond temporal resolutions are achieved.

While the pulsed laser illuminates the tissue at 20 MHz, the SwissSPAD2 camera shifts its gated window every delay time (minimal 18 ps) over maximal 200 gate steps, thus collecting photons at different depths inside the target tissue volume (FIG. 2A). Specifically, after acquiring a first gated image at the shallowest depth, the gate shifts one delay time to the next step with respect to the reference from the laser pulse. This is illustrated by the arrows extending from each step, where at step 2, there is a 10X delay; at step 3, there is a 20X delay; at step 4, there is a 30X delay; at step 5, there is a 40X delay; at step 6, there is a 50X delay; and at step 7, there is a 60X delay. Data collections are then repeated over up to 200 gates until the final gate position is reached (i.e., 200th gate at the deepest depth). For illustrative clarity in FIG. 2A, only 7 gate positions within a time interval of 10 ns (10×1 ns delay time) are illustrated instead of the full 200 gates.

Before data acquisition, the offset time is determined to ensure that the first gated image is taken from the tissue surface (i.e., the shallowest depth). The offset is defined as the number of gates to be skipped before useful data acquisition (e.g., from tissue surface) begins. The offset time depends on MTR-LSCI instrument configuration such as laser and camera synchronization time, light speed and working distance. To determine the offset time, the SwissSPAD2 camera captured 200 gated images with a delay time of 1 ns over a total of 200 ns (FIG. 2B). Based on the gate intensity profile taken from a calibration tissue phantom, offsets between 30-32 ns (shaded region in FIG. 2A) were used in a typical MTR-LSCI setup. For fair comparisons, all experiments in tissue phantoms and animals utilized the constant offset time of 30.5 ns.

After determining the offset time, data acquisition in designed experiments can start with the minimal delay time of 18 ps over 200 gates to maximize depth sensitivity of MTR-LSCI measurements (FIG. 2C). As a result, the total detection time window over 200 gates was 3.6 ns (200×18 ps).

To collect one binary frame image at each gate step, the field-programmable gate array (FPGA) implementation in the SwissSPAD2 program involves opening the camera 400 times for data acquisition. The relation between the number of binary frames (Y) and the bit-depth (b) is defined as Y=2^{b}−1. However, since 10-bit images are constructed from four individually saved 8-bit images, Y is equal to 1,020 in 10-bit mode (b=10), instead of 1,023. Therefore, a total of 1020 binary frames are integrated in the FPGA to construct one 10-bit image at each gate step within ˜20 ms (1020×400×50 ns). During this acquisition time (˜20 ms), the total active exposure time for one 10-bit image is ˜5.3 ms (1020×400×13.1 ns). As such, the total time to acquire and save one 10-bit image to a hard drive in the computer is ˜30 ms (20 ms for data collection plus 10 ms for data readout and storage).

The frame sampling time of 30 ms is equivalent to a frame rate of ˜33 Hz. However, the actual highest frame rate in the experiments was only ˜0.2 Hz. The actual SwissSPAD2 frame rates depend on multiple factors including binary frame readout, exposure time (acquisition time) and number of acquired gates. Since the SwissSPAD2 camera operates in global shutter mode, the sensor is insensitive to photons during read-out. Also, data acquisition must be stopped when image data are transferred from the RAM to the hard drive in the computer. Lastly, data acquisition is currently performed through 32-bit MATLAB code, which is not optimal for fast sampling and slower than 64-bit MATLAB. Future optimization of the SwissSPAD2 operating system with new editions of Python code or C++ would contribute to improving the frame rate. Additionally, incorporating a powerful computer equipped with a rapid SSD drive can promote data read and write speeds.

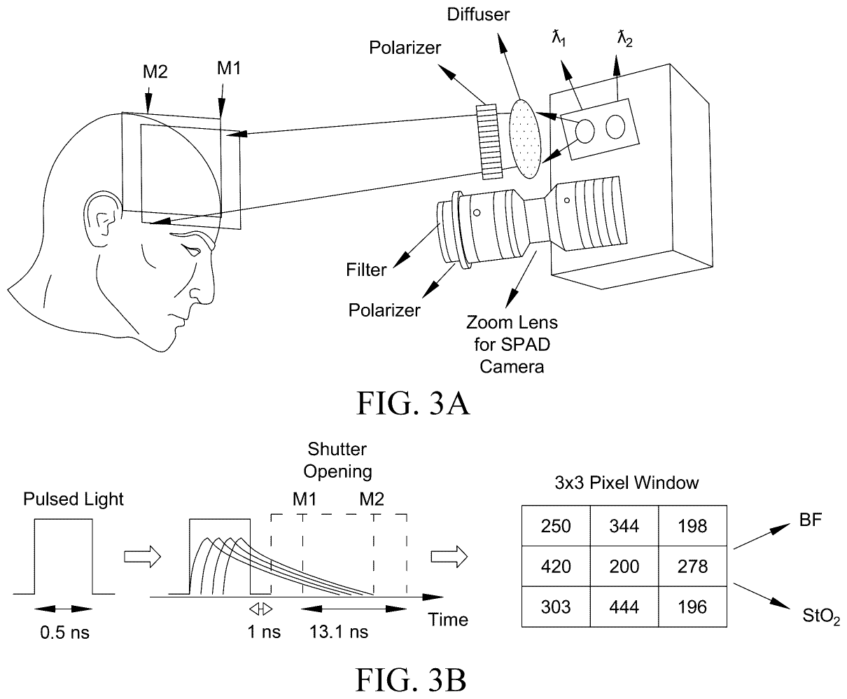

FIGS. 3A and 3B show the principle and data acquisition of MTR-LSCI with the gated mode enhancing the depth sensitivity. One improvement in the MTR-LSCI according to embodiments of the present invention versus single-wavelength MTR-LSCI is its ability to apply gated strategies to multiple wavelengths for data acquisition of both BF and STO₂ at different depths of the tissue.

The MTR-LSCI employs nanosecond or picosecond pulsed NIR lasers at different wavelengths, such as λ₁=785 nm and λ₂=830 nm. Alongside these lasers, a time-gated camera serves as a 2D detector array. This combination enables the simultaneous acquisition of diffuse laser speckle fluctuations for imaging BF and the light intensities (I_{λ})) at multiple wavelengths (λ≥2) for mapping concentrations of oxy-hemoglobin [HbO₂] and deoxy-hemoglobin [Hb], thereby providing a multimodal capability.

Crucially, by implementing time-gated strategies in MTR-LSCI measurements, the system, according to embodiments of the present invention, distinguishes between short and long photon paths through the layered head tissues, including scalp, skull and brain. This differentiation allows for quantitatively assessing BF, [HbO₂], and [Hb] noninvasively in different tissue types of both animals and humans, accommodating different tissue size scales and providing multiscale functionality.

### MTR-LSCI Data Processing for BF and StO₂Maps

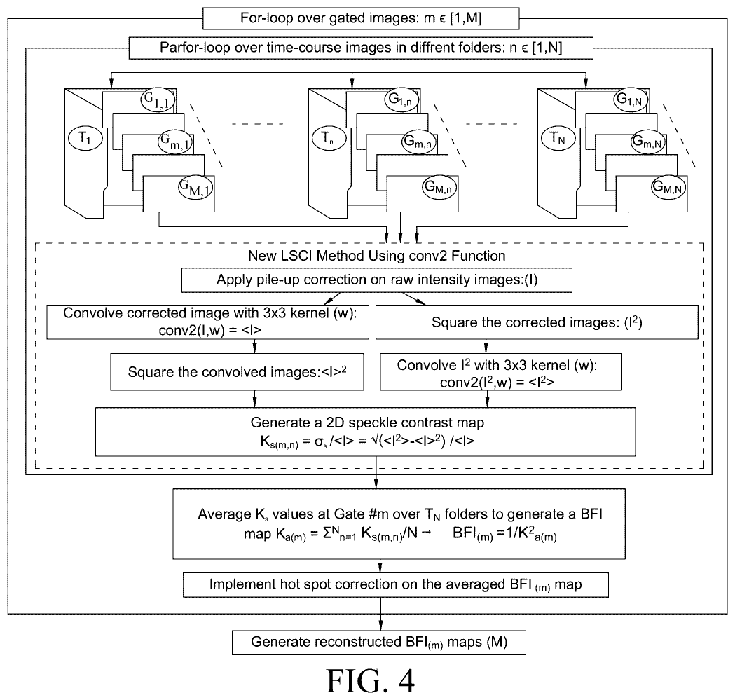

FIG. 4 illustrates multiple steps using algorithms, according to embodiments of the present invention, with parallel computation and convolution functions, in MATLAB, to rapidly process single-wavelength MTR-LSCI data and generate 2D flow maps. Specifically, the SwissSPAD2 camera captures diffuse laser speckle fluctuations resulting from motions of red blood cells in target tissue volume, measured at different depths with different gates. The resulting gated images at different depths numbered from G1 to GM are stored sequentially to a folder in the computer. The continuously collected time-course gated images are stored in different folders, numbered from T1 to TN. Two nested for-loops are used to process the gated and time-course images, respectively. The outer for-loop (solid outer box) processes the gated images (Gm) sequentially. By contrast, the inner parfor-loop (solid inner box) from MATLAB Parallel Computing Toolbox processes multiple time-course images (Tn) simultaneously, which significantly reduces the computation time.

The dashed box in FIG. 4 shows a new fast LSCI method that uses 2D convolution functions from MATLAB Image Processing Toolbox to convert an intensity image obtained with intensity or gated mode to a 2D flow map. Specifically, a pile-up correction first applies on the intensity image as a pre-processing step to correct possible deformations of the decay shape in the timing and intensity of photon events. The following correction formula was used to adjust for pile-up effect:

$${I}_{cor} = log ( 1 - \frac{{I}_{rec}}{{I}_{max}} ) ,$$

where Imax is the maximum photon count (1024 for a 10-bit image), I_{rec} is the recorded photon count and I_{cor} is the corrected photon count.

After pile-up correction, the corrected gated image was converted to a speckle contrast image based on LSCI analysis:

$${K}_{s} = \frac{{σ}_{s}}{〈 I 〉} = \frac{\sqrt{〈 {I}^{2} 〉 - {〈 I 〉}^{2}}}{〈 I 〉} ,$$

where K s is defined as the ratio of the standard deviation to mean intensity in a pixel window (w=Nₚᵢₓₑₗₛ×Nₚᵢₓₑₗₛ). A pixel window size of 3×3, 5×5 or 7×7 is usually used to balance the detection sensitivity and spatial resolution. Based on the experimental data, Kₛ was quantified over an optimal window of 9 pixels (i.e., Nₚᵢₓₑₗₛ=3).

Conventional methods for LSCI analysis use two nested for-loops to iteratively compute Kₛ values through all pixels, which is time consuming. By contrast, the LSCI algorithm, according to embodiments of the present invention, performs the conv2 function in MATLAB with different kernels to obtain 2D matrixes of <I>, <I²>, and <I>², without the need of time-consuming for-loops. A 2D matrix of Kₛ is then generated from these convolutions. Multiple time-course K s maps over a certain period (e.g., T_{N}) are averaged to generate one Kₐ map with improved SNR. Although the exact relationship between the Kₛ (Kₐ) and flow is nonlinear, blood flow index (BFI) can be approximated as the inverse square of the speckle contrast in LSCI analysis

$$( BFI \sim \frac{1}{{K}_{s}^{2}} ) .$$

The final step was the correction of hot pixel spots in the reconstructed BFI map. Because of flaws in the manufacturing process, a small fraction of the camera pixels exhibits elevated dark counts (typically 10 or 100 times higher than the median dark count rate), corresponding to the so-called “hot pixels”. The hot pixels are distributed randomly across the camera sensor array, appearing as high-intensity dots (artifacts) on the collected image. Initially, one approach sought to mitigate the hot pixels from raw intensity images prior to the BFI reconstruction process. However, this preliminary endeavor was only partially successful, with traces of the hot pixels persisting despite the correction. In addition, this preprocessing approach incurred significant computational overhead. Consequently, a post-reconstruction strategy was used, wherein the hot pixel locations were identified and corrected from the reconstructed BFI map. To fill in the missing values corresponding to the hot pixels, their locations were first tagged and a median filter was applied iteratively only on each tagged pixel of the reconstructed BFI map. In this way, the median filter did not affect normal pixels as they were masked out. Experimentally, it was found that five iterations are usually enough to correct the hot pixels.

The final outputs from the outer for-loop and inner parfor-loop are gated BFI maps (M). The implementation of parallel computation using parfor-loop (solid inner box) and matrix convolutions in the new LSCI analysis (dashed box) significantly shortened the time for reconstructing BFI maps. In this study, MATLAB (R2021a) codes run on a desktop computer equipped with the Intel(R) Xeon (R) W-2245 CPU (8 Cores) operating at a frequency of 3.90 GHz. MATLAB uses 8 CPU cores as 8 co-workers for executing parallel computations. When analyzing mouse data consisting of 400 folders (T400) with 80 gated images (G80) in each folder, the total time required to reconstruct 80 gated BFI maps (including the averaging over 400 folders) was reduced significantly from -68 hours to 115 seconds. More specifically, the time to reconstruct one gated BFI map was reduced from ˜50 minutes to -5 seconds.

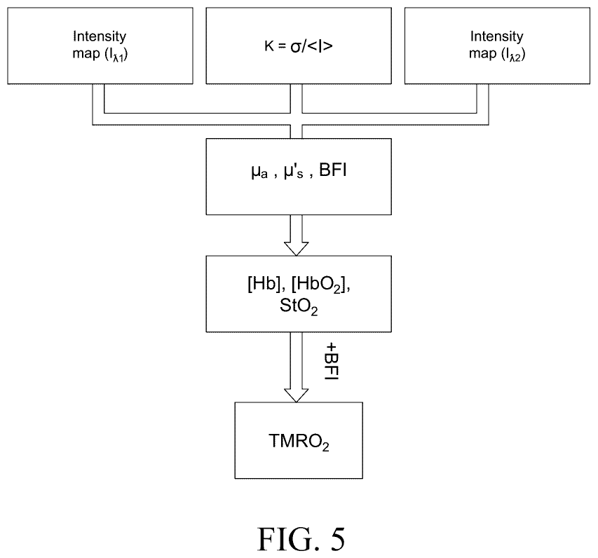

FIG. 5 illustrates multiple steps to process MTR-LSCI data and generate 2D

maps of StO₂ and TMRO₂ variations. StO₂ variations are obtained from the measured light intensity changes at the two wavelengths using the modified Beer-Lambert law:

$$Δ {μ}_{a} ( λ ) = \frac{ln ( \frac{{I}_{λ B}}{{I}_{λ T}} )}{{DPF}_{λ}} \\ Δ [ {Hb O}_{2} ] = \frac{{ε}_{Hb} ( {λ}_{1} ) Δ {μ}_{a} ( {λ}_{2} ) - {ε}_{Hb} ( {λ}_{2} ) Δ {μ}_{a} ( {λ}_{1} )}{{ε}_{Hb} ( {λ}_{1} ) {ε}_{{HbO}_{2}} ( {λ}_{2} ) - {ε}_{{HbO}_{2}} ( {λ}_{1} ) {ε}_{Hb} ( {λ}_{2} )} \\ Δ [ Hb ] = \frac{{ε}_{{HbO}_{2}} ( {λ}_{2} ) Δ {μ}_{a} ( {λ}_{1} ) - {ε}_{{HbO}_{2}} ( {λ}_{1} ) Δ {μ}_{a} ( {λ}_{2} )}{{ε}_{Hb} ( {λ}_{1} ) {ε}_{{HbO}_{2}} ( {λ}_{2} ) - {ε}_{{HbO}_{2}} ( {λ}_{1} ) {ε}_{Hb} ( {λ}_{2} )} ,$$

where Δμ_{α}(λ) is the relative change of absorption coefficient μ_{α} at wavelength λ(λ₁=785 nm and λ₂=830 nm). The ε_{Hb}(λ) and ε_{HbO}_{₂}(λ) are the extinction coefficients of Hb and HbO₂. The I_{λB} and I_{λT} are the measured light intensities at the baseline and at time T, respectively. DPF_{λ} is the differential path factor. Combinations of these parameters yield tissue total hemoglobin concentration ([tHb]=[Hb]+[HbO₂]) and tissue blood oxygen saturation StO₂=([HbO₂]/tHb)×100%.

The relative change in TMRO₂ can be calculated based on Fick's law,

$${rTMR O}_{2} = rBFI \times \frac{[ 1 - {St O}_{2} ]}{[ 1 - {St O}_{2 base ]}}$$

where rBFI is relative blood flow index and StO_{2base} is the baseline StO₂ before physiological changes.

### Depth Sensitivity Validation of MTR-LSCI Using Head-Simulating Phantoms

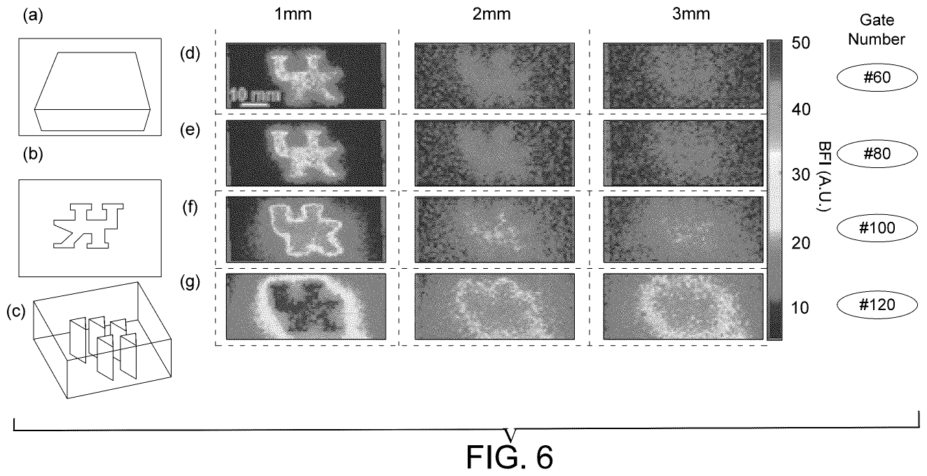

Referring to FIG. 6, head-simulating phantoms with known optical property and geometry (sections (a) through (c)) were fabricated and used to illustrate the fundamental concept and depth sensitivity of MTR-LSCI. The solid phantoms have a top layer with varied thickness to mimic the skulls of small animals (rodents). Three solid phantoms (no flow) with the empty channels bearing the University of Kentucky logo were fabricated using a 3-D printer (SL1, Prusa) with a top layer thicknesses of 1, 2 and 3 mm, respectively. The solid phantoms were made of Titanium dioxide (TiO₂), India ink (Black India, MA) and clear resin (eSUN Hard-Tough). The empty UK logo channels were then filled with liquid solutions composed of Intralipid particles (Fresenius Kabi, Sweden), India ink and water. India ink concentration in the phantoms regulates the absorption coefficient μₐ while TiO₂ and Intralipid concentrations regulate the reduced scattering coefficient μ′ₛ. Intralipid particles in the liquid phantom provide Brownian motions (i.e., particle flow) to mimic movements of red blood cells in the brain. Optical properties of both solid and liquid phantoms were set in the range of realistic tissue: μₐ=0.03 cm⁻¹, and μ′ₛ=9 cm⁻¹.

The noncontact MTR-LSCI system was configured to image the ROI of 30 x 60 mm 2 on the UK logo phantom surface. MTR-LSCI collected 200 gated images at different depths sequentially with an interval delay time of 18 ps. Flow contrasts of UK logo phantoms at varied gates/depths were analyzed to demonstrate the depth sensitivity and spatial resolution of MTR-LSCI (FIG. 6, sections (d) through (g)).

### In Vivo Study Protocols

All animal experimental procedures were approved by the UK Institutional Animal Care and Use Committee (IACUC). One mouse and six rats were imaged to assess the performance of single-wavelength MTR-LSCI for mapping BFI at different depths. An adult male, C57BL6 mouse (9 months old) was anesthetized with Isoflurane and imaged with both gated and intensity modes to compare imaging spatial resolutions (vessel visualization) as the mouse has a thinner skull than the rat. Six rats were imaged with the gated mode to show the capability of MTR-LSCI for 2D mapping of BFI in deeper brain through a thicker skull. Both the mouse and rats were subjected to acquisition of 80 gated images using a constant gate delay time of 18 ps. Eighty gated images (instead of 200) were taken in animal studies to achieve a sampling rate higher than for the phantom experiments. The relative time-course changes in CBF (rCBF) were calculated by normalizing BFI data to the baseline value before the hypercapnia stimuli or cerebral ischemic challenge.

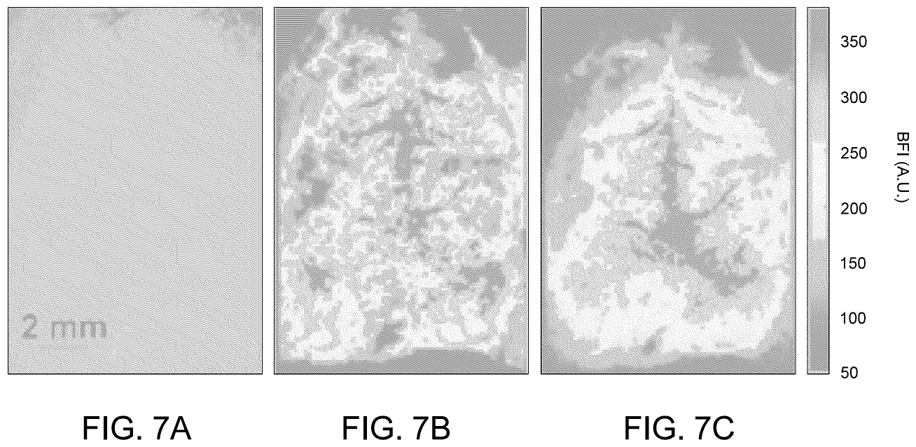

FIGS. 7A through 7C show the baseline imaging of BFI in a mouse. The mouse on the heating blanket was anesthetized (1-2% isoflurane) with its head secured on a stereotaxic frame. The mouse scalp was surgically removed to reduce its partial volume effect on the deep brain. The noncontact MTR-LSCI was positioned above the mouse head and scanned over a ROI of 20×10 mm² on the exposed intact skull.

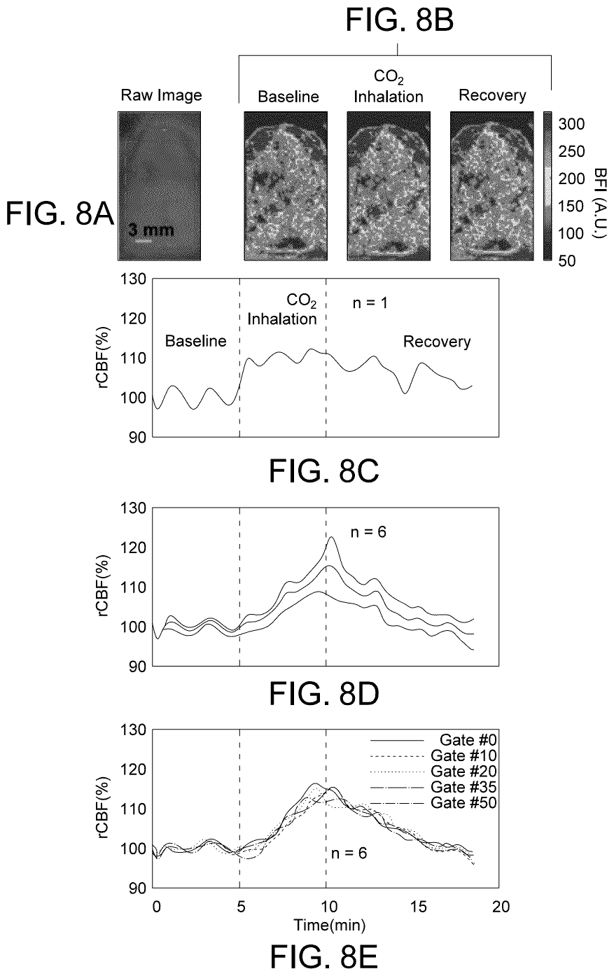

FIGS. 8A through 8E illustrate the continuous imaging of global rCBF increasing in rats during CO₂ inhalations. MTR-LSCI imaging was continuously performed in six adult male Sprague-Dawley rats (2-3 months) before, during and after the exposure to a gas mixture consisting of 8% CO₂ and 92% O₂. It is well known that CO₂ is a vascular dilator, leading to a global increase in CBF. The rat on the heating blanket was anesthetized (1-2% isoflurane) with its head secured on a stereotaxic frame. The rat scalp was surgically removed to expose its skull. The noncontact MTR-LSCI scanned over a ROI of 30×15 mm² on the exposed intact skull for continuous BFI mapping. After a baseline measurement for ˜5 minutes, the mixed gas of 8% CO₂/92% O₂ was administered through a nose cone connected to Matheson Mixer Rota-meter for ˜5 minutes. The CO₂ was then stopped, and MTR-LSCI measurements lasted for ˜10 minutes to record BFI recovery towards baseline.

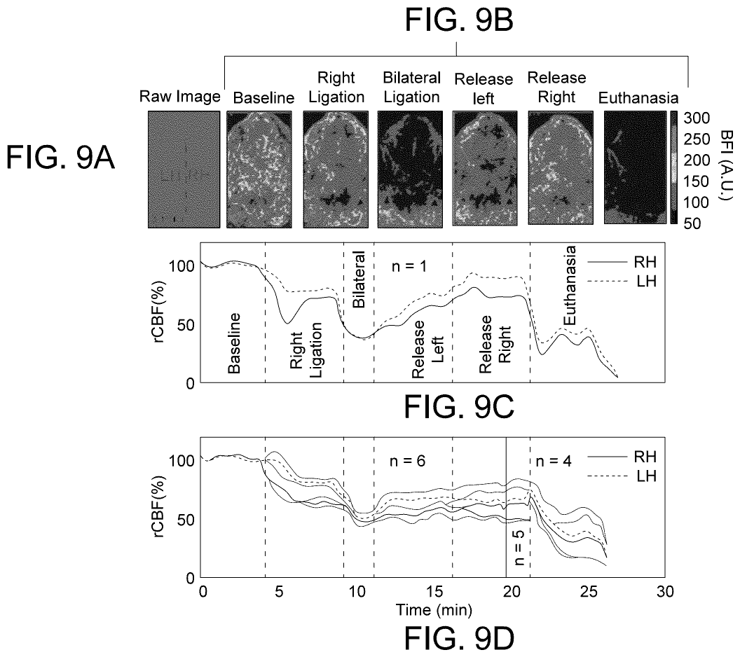

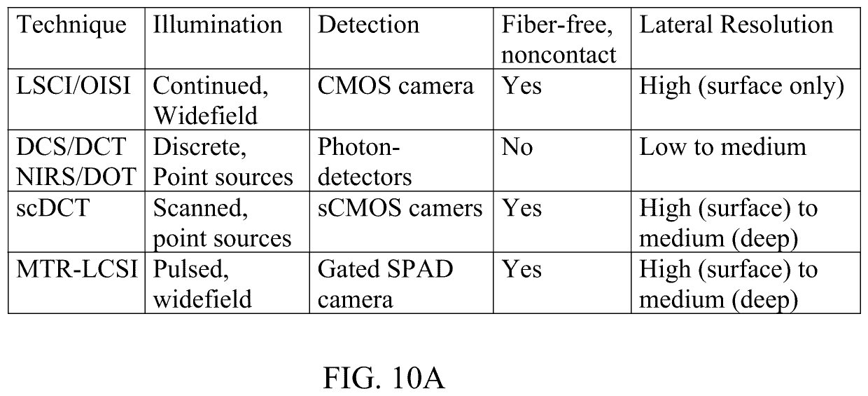

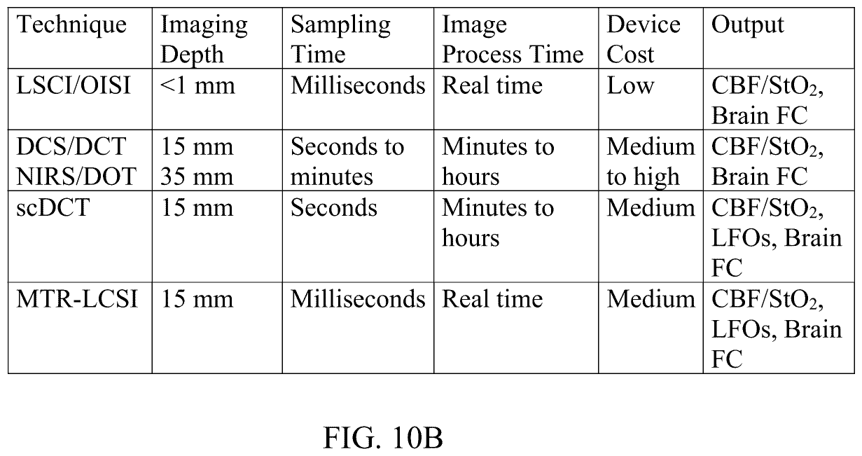

FIGS. 9A through 9D illustrate continuous imaging of regional rCBF decreasing in rats during transient carotid artery ligations. After the CO₂ -inhalation experiments, the six rats underwent transient bilateral ligations of common carotid artery (CCA) to create sequential decreases in CBF within the left and right hemispheres. The hairs at cervical surgical site for CCA ligations were shaved and removed with hair cream, and cervical skin was disinfected with Betadine followed by 70% Ethanol. A midline incision was performed to expose and isolate both the left and right CCAs. A sterile surgical suture was wrapped around each CCA, and a loose knot was tied on each suture without impeding blood flow. MTR-LSCI configuration and continuous measurements were identical to these described in the CO2 inhalation protocol. After a baseline measurement for ˜5 minutes, the right CCA knot was tightened for ˜5 minutes to occlude the right CCA. Then the left knot was tightened for ˜2 minutes to induce a transient global ischemia. The left and right knots were then released sequentially for ˜5 minutes each, allowing the restoration of CBF to the brain. At the end, the animal was euthanized with 100% CO2 inhalation.

### Statistical Analysis

Statistical analyses in animal studies were conducted using SPSS software (version 29). Differences in rCBF variations across different phases of stimuli (CO₂ inhalations and transient arterial occlusions) were evaluated using repeated measures analysis of variance (ANOVA). A p-value <0.05 is considered significant for statistical analyses.

### Results

MTR-LSCI Enables Detection of Flow Contrasts with Depth Sensitivity

FIG. 6, sections (d) through (g), show resulting 2D maps of particle flow contrasts captured at the gates of #60, #80, #100, and #120 from three UK logo phantoms with the top layer thicknesses of 1, 2 and 3 mm, respectively. One hundred (100) time-course images were taken and averaged at each gate to increase SNRs. Higher flow contrasts were observed at deeper depths with larger gate numbers, indicating the depth sensitivity of imaging by the MTR-LSCI. SNRs decreased with the increase of imaging depth (i.e., from Gate #60 to Gate #120) and top layer thickness (i.e., from 1 to 3 mm). These results are expected as deeper penetration and thicker top layer resulted in fewer diffused photons being detected, thus leading to lower SNRs. Results obtained from phantoms indicate that each gate delay of 18 ps corresponds to a 63 μm depth increment.

### MTR-LSCI Gated and Intensity Modes Generate Similar BFI Maps in the Mouse

FIG. 7A shows a raw intensity image of the mouse skull with its scalp retracted. FIG. 7B shows the reconstructed BFI map with the gated mode at Gate #0. Gate #0 was selected for the comparison with the intensity mode sensitive to tissue surface. FIG. 7C shows the reconstructed BFI map with the intensity mode. The camera exposure time for the intensity mode was 5.12 ms. Four hundred (400) time-course images were taken and averaged for both gated and intensity modes to increase SNRs. The gated and intensity modes generated similar BFI maps. However, the gated image (FIG. 7B) provided more vasculature details than the intensity image (FIG. 7C), which is likely due to depth sensitivity of the gated mode.

MTR-LSCI Captures rCBF Increases During CO2 Inhalations in Rats

FIG. 8A shows a raw intensity image of the rat skull with its scalp retracted. FIG. 8B shows BFI maps before, during, and after CO₂ inhalation, taken by the MTR-LSCI at Gate #35 from an illustrative rat (Rat #4). Gate #35 was selected to ensure sufficient penetration depth to image the rat brain. Based on rat head anatomy, a penetration depth of 2 mm reaches the rat brain cortex through its skull (thickness of −1 mm). Since a gate delay of 18 ps is equivalent to −63 μm depth traveling inside biological tissues, imaging depth at Gate #35 exceeds 2 mm (i.e., 35×63 μm). Approximately 70 time-course images were averaged during the baseline and CO₂ inhalation phases respectively to generate BFI maps with improved SNRs. Approximately 140 images were averaged during the recovery phase. FIG. 8C shows time-course rCBF changes in the selected ROI in the same illustrative rat (Rat #4), measured continuously by the MTR-LSCI with a sampling rate of 0.2 Hz. rCBF changes were calculated by normalizing BFI data to the baseline values prior to CO₂ inhalation. FIG. 8D shows average time-course changes in rCBF over 6 rats. Average time-course changes in rCBF and p-values for comparing those changes at different phases of CO₂ inhalation are summarized in Table 1 and Table 2, respectively. The inhalation of 8% CO 2 resulted in a significant increase in rCBF at the endpoint of inhalation compared to the baseline (mean ±standard error: 112.3% ±3.8%; repeated measures ANOVA: p=0.026), which is consistent with previous studies. A significant difference (repeated measures ANOVA: p=0.042) was also observed between the 8% CO₂ inhalation and the endpoint of the recovery phase. FIG. 8E shows average time-course rCBF changes taken at different gates (Gate#1, Gate #10, Gate #20, Gate #35, Gate #50) over 6 rats. No significant differences in rCBF were found among different gates.

TABLE 1
Average rCBF changes (mean ± standard error) from their baselines
(100%) during CO₂ inhalation over 6 rats

| | 8% CO₂ inhalation | Recovery |
|---|---|---|
| Brain Region | 112.3% ± 3.8% | 99.5% ± 2.3% |

TABLE 2
Repeated measures ANOVA to assess differences in rCBF changes
between different stages of CO₂ inhalation

| | | p-value |
|---|---|---|
| Baseline | 8% CO₂ Inhalation | 0.026* |
| | Recovery | 0.852 |
| 8% CO₂ | Baseline | 0.026* |
| Inhalation | Recovery | 0.042* |
| Recovery | Baseline | 0.852 |
| | 8% CO₂ Inhalation | 0.042* |

*The difference between mean values is significant: p < 0.05.

MTR-LSCI Captures rCBF Decreases During Transient Artery Ligations in Rats

FIG. 9A shows a raw intensity image of the rat skull which was used to generate BFI map. FIG. 9B shows BFI maps before, during, and after transient global ischemia, induced by unilateral and bilateral CCA ligations, and during 100% CO₂ euthanasia on an illustrative rat (Rat #1). BFI maps were taken by the MTR-LSCI at Gate #35. Approximately 70 images were averaged for almost all phases of ligations to improve SNRs, except the transient bilateral ligation period where 28 images were averaged. FIG. 9C shows time-course rCBF changes in the same illustrative rat (Rat #1) at the left and right hemispheres, measured continuously by MTR-LSCI with a sampling rate of 0.2 Hz. rCBF changes were calculated by normalizing BFI data to the baseline values prior to CCA ligations. FIG. 9D shows average time-course rCBF changes at two hemispheres over 4-6 rats. The number of rats for averaging varied due to the unfortunate death of two rats towards to the later phases of experiments. Table 3 and Table 4 provide a summary of the average changes in rCBF and p-values for comparing those changes during various phases of CCA ligations. As expected, sequential ligations of the right and left CCAs, followed by their releases, led to significant changes in rCBF in corresponding hemispheres compared to their baselines (repeated measures ANOVA: p<0.05). No significant differences in rCBF were observed among different gates (data are not shown).

Additionally, the euthanasia phase was examined to include all available data (n=4). Paired t-test was employed to evaluate the difference in rCBF between the baseline and euthanasia phase. Apparently, euthanasia resulted in further reduction in rCBF. During 100% CO₂ euthanasia, rCBF values were reduced significantly to 37.6% ±10.4% (p=0.009) and 43.1% ±15.3% (p=0.034) in right and left hemispheres, respectively (Table 3). Notably, rCBF was reduced to the minimal values at the endpoint of 100% CO₂ euthanasia, with the values of 29% ±15.6% and 33% ±19.3% in the right and left hemispheres, respectively. During the 100% CO₂ procedure, one of the four rats exhibited a remarkable resistance to the inhalation and did not succumb to euthanasia. As a result, this rat did not show an obvious decrease in rCBF, leading to a bias in the overall estimation of group rCBF reduction.

TABLE 3
Average rCBF changes (mean ± standard error) from their baselines
(100%) during unilateral and bilateral CCA ligations over 4 to 6 rats

| | | | Release | Release Right | |
|---|---|---|---|---|---|
| | Right | Bilateral | Left | Ligation | |
| | Ligation | Ligation | Ligation | (Recovery) | Euthanasia |
| | n = 6 | n = 6 | n = 6 | n = 6 | n = 4 |
| Right | 66.7% ± | 51.1% ± | 53.4% ± | 59.7% ± 10% | 37.6% ± |
| Hemisphere | 1.8% | 2.4% | 3.9% | | 10.4% |
| (RH) | | | | | |
| Left | 84.4% ± | 53.8% ± | 64.7% ± | 65.6% ± 11.7% | 43.1% ± |
| Hemisphere | 3.1% | 4.3% | 5.5% | | 15.3% |
| (LH) | | | | | |

TABLE 4

| | | Right | Left |
|---|---|---|---|
| | | Hemisphere | Hemisphere |
| | | (RH) | (LH) |
| | | p-value | p-value |
| Baseline | Right Ligation | <0.001* | <0.001* |
| | Bilateral Ligation | <0.001* | <0.001* |
| | Release Left | <0.001* | <0.001* |
| | Ligation | | |
| | Release Right | 0.004* | 0.010* |
| | Ligation | | |
| | (Recovery) | | |
| Right | Baseline | <0.001* | <0.001* |
| Ligation | Bilateral Ligation | <0.001* | <0.001* |
| | Release Left | 0.028* | 0.004* |
| | Ligation | | |
| | Release Right | 0.569 | 0.145 |
| | Ligation | | |
| | Recovery) | | |
| Bilateral | Baseline | <0.001* | <0.001* |
| Ligation | Right Ligation | <0.001* | <0.001* |
| | Release Left | 0.650 | 0.046* |
| | Ligation | | |
| | Release Right | 0.462 | 0.319 |
| | Ligation | | |
| | (Recovery) | | |
| Release | Baseline | <0.001* | <0.001* |
| Left | Right Ligation | 0.028* | 0.004* |
| Ligation | Bilateral Ligation | 0.650 | 0.046* |
| | Release Right | 0.438 | 0.914 |
| | Ligation | | |
| | (Recovery) | | |
| Release | Baseline | 0.004* | 0.010* |
| Right | Right Ligation | 0.569 | 0.145 |
| Ligation | Bilateral Ligation | 0.462 | 0.319 |
| (Recovery) | Release Left | 0.438 | 0.914 |
| | Ligation | | |

*The difference between mean values is significant: p < 0.05

### Discussion and Conclusions

Continuous and longitudinal monitoring of CBF holds significance for both neuroscience research and clinical applications. In contrast to large, expensive, and single-shot neuroimaging modalities, such as MRI and CT, portable and affordable optical imaging instruments enable continuous and longitudinal assessment of CBF at the bedside of clinics. Conventional continuous-wave systems with coherent laser illumination for CBF measurements include LSCI and DCS; both have limitations. LSCI with a widefield illumination and 2D ordinary camera detection enables fast and high-resolution 2D mapping of superficial CBF (depth less than 1 mm), whereas DCS with point sources and discrete photodetectors enables low-resolution 3D tomography of deeper CBF (depth up to centimeters). Moreover, collected cerebral signals by LSCI and DCS are inherently influenced by partial volume artifacts from overlayer tissues (scalp and skull). More recently, time-resolved systems such as TR-DCS use pulsed point sources and discrete single-photon avalanche diodes to measure temporal autocorrelation functions for quantifying CBF at different depths (i.e., depth sensitive). However, most TR-DCS systems suffer from poor spatial resolution and limited head coverages due to limited numbers of discrete sources and detectors.

An innovative MTR-LSCI technique, according to embodiments of the present invention, can enable fast and high-resolution 2D mapping of BF and StO2 at different depths of target tissue. MTR-LSCI illuminates picosecond-pulsed, coherent, widefield NIR light at different wavelengths onto the tissue and synchronizes a fast, time-gated, SPAD camera to map BF and StO₂ distributions at different depths. Similar to LSCI, integration of widefield illumination and 2D camera detection in MTR-LSCI enables fast and high-resolution 2D mapping of BF and StO₂. By applying the time-resolved (TR) method in brain studies, for example, MTR-LSCI discards photons with short pathlengths, which predominantly traverse extracerebral layers such as the scalp and skull. Meanwhile, photons with longer pathlengths are retained, which penetrate deeper and ultimately reach the brain.

To prove the concept, MTR-LSCI systems, such as that shown in FIG. 1, were developed, for the first time, and optimized iteratively. Adjustable optomechanical holders were utilized to install and align the free space pulsed lasers and SwissSPAD2 camera on the same ROI. An articulating baseball stage was employed for easy rotation of the light sources. All components of the portable MTR-LSCI system were consolidated within a compact probe, affixed to a 360° holder, simplifying alignment with a chosen FOV/ROI. At least two diffusers were placed in front of the point pulsed sources to achieve homogeneous widefield illumination. A pair of polarizers were added across the source and detection paths to reduce the influence of source reflection directly from the tissue surface. A long-pass filter was used in the detection path to minimize the influence of ambient light.

To synchronize the lasers and camera, the picosecond pulsed lasers or a signal generator as the master device provided the timing reference for the operation of the SwissSPAD2 at 20 MHz or any other ranges from 10-80 MHz (FIGS. 2A through 3B). The customized control software (MATLAB) allowed for setting up multiple camera parameters used in each experiment, including initial offset time, gate delay, number of gates and exposure time. Both the offset time and gate delay played a role in determining imaging depth and sensitivity. By adjusting the number of gates to be collected, the sampling rate was also changed. For example, collecting 4 gates versus 80 gates achieved sampling rates of 1 Hz and 0.2 Hz, respectively. On the other hand, the optimization of laser power was essential to adapt the linearity range of SwissSPAD2 camera and comply with the ANSI standard.

To expedite data processing, new algorithms with parallel computation and convolution functions in MATLAB were developed for processing MTR-LSCI data and generating 2D flow and oxygenation maps rapidly (FIG. 4 and FIG. 5). Conventional LSCI algorithms consider the subdivision of an original image into multiple pixel windows (Nₚᵢₓₑₗₛ×Nₚᵢₓₑₗₛ), which are computationally extensive due to the use of multiple nested for loops. This bottleneck was addressed by leveraging the conv2 function in combination with the parallel processing capabilities of multicore calculations in MATLAB. As a result, the reconstruction time to generate a single gated BFI map was reduced remarkably from 50 minutes to only 5 seconds.

To demonstrate the depth-sensitivity of MTR-LSCI, unique head-simulating phantoms were designed and fabricated with the top solid layer representing the skull (zero flow) and underneath layer of UK logo filled with Intralipid particle flow (non-zero flow) representing the brain (FIG. 6). The top layer thicknesses of 1-3 mm coincide approximately with the skull thicknesses of mice and rats. Results from the phantom measurements illustrate the capability of MTR-LSCI for 2D mapping of flow contrasts in the deep “brain” through the surface “skull”.

The analysis of the data from the phantom with 3 mm top layer revealed the emergence of the “UK logo” shape around the gate number of 95 (FIG. 6, section (f)). Considering that light travels approximately 6 mm into and out of the top layer, this corresponds to an approximate depth increment of ˜63 μm for each gate delay (i.e., 6 mm/95 gates). This finding aligns with previous depth estimation using SwissSPAD2 for subsurface fluorescence imaging, where a linear relationship depending on tissue reduced scattering coefficient (μ′ₛ) was found between the photon flight time and penetration depth. With the gate delay of 18 ps, a depth increment of ˜360 μm was observed in the tissue phantom with μ′ₛ=45 cm⁻¹. After adjusting the difference in the tissue phantom (9 cm⁻¹, five times smaller), each gate delay of 18 ps corresponds to a depth increment of ˜72 μm (i.e., 360 μm/5).

To assess the capability of MTR-LSCI in capturing cerebral vasculature, in vivo mapping of BFI was performed in a mouse with the thinner skull (in contrast to rats) using both gated and intensity modes (FIGS. 7A through 7C). The image close to the surface (Gate #0) shows the vascular network with apparent anatomical landmarks revealing more details in the gated mode, as compared to the intensity mode. It should be noted that MTR-LSCI with the intensity mode is equivalent to the conventional LSCI for mapping superficial CBF. Based on the scale of mouse brain microvasculature, MTR-LSCI achieved a spatial resolution of tens of micrometers on the tissue surface (FIGS. 7A through 7C). To assess the capability of MTR-LSCI in continuous monitoring of global/regional rCBF changes, a group of adult rats were measured during transient cerebral hypocapnia (via CO₂ inhalation for 5 minutes) and transient cerebral ischemia (through CCA ligations) (FIGS. 8A through 9D). During 8% CO₂ inhalation, rCBF increased significantly from 100% baseline to 112.3% ±3.8% (Table 1 and Table 2). The results agree fairly well with a previous study in adult rats utilizing speckle contrast diffuse correlation tomography (scDCT) with 3D reconstruction: from 100% baseline to 119% ±8% during 10% CO2 inhalation for 10 minutes. The small discrepancy in rCBF increase between the two studies can likely be attributed to the differences in CO₂ concentrations (8% versus 10%) and CO₂ inhalation duration (5 minutes vs 10 minutes).

The sequential CCA ligations induced significant decreases in rCBF: from 100% baseline to 51.1% ±2.4% and 53.8% ±4.3% within the right and left hemispheres, respectively (Table 3 and Table 4). Previously using scDCT, it was found that sequential CCA ligations caused significant rCBF reductions from 100% baseline to 34% ±10% and 32% ±11% in the right and left hemispheres, respectively. The discrepancy in rCBF reductions between the two studies may be attributed to the difference in unilateral CCA ligation durations (5 minutes vs 10 minutes). As expected, 100% CO₂ euthanasia resulted in significant reductions in rCBF at the endpoint: 29% ±15.6% and 33% ±19.3% of their baselines in the right and left hemispheres, respectively. Also, MTR-LSCI achieved a spatial resolution of 1-2 millimeters when mapping the deep brain of adult rats (FIG. 8A through FIG. 9D).

No significant differences in rCBF responses were observed among different gates during both CO₂ inhalations (FIG. 8E) and CCA ligations. An underlying impact factor could be the large gate width (13.1 ns) of the SwissSPAD2 camera used in the MTR-LSCI system, leading to the detection overlap of early and late photons. The presence of this phenomenon resulted in the overlap of different sample volumes/depths, leading to a decrease in depth sensitivity. The depth sensitivity can be improved by using a newly developed gated camera, SPAD512S, with an achievable gate width of 6 ns. A customized SPAD512S camera with integrated microlenses can be used to increase the effective fill factor from 10% to 50%, thus improving the imaging sensitivity by a similar amount (3-4 fold). Also, the SPAD512S with a new operating system (C++) is expected to lead to a higher sampling rate (up to 15 Hz), compared to the SwissSPAD2 (up to 1 Hz). Increasing the sampling rate opens up the possibility for mapping brain functional connectivity. Furthermore, the inclusion of other picosecond-pulsed lasers operating at different NIR wavelengths would enable simultaneous imaging of CBF and ScO2 distributions using NIRS principles.

In conclusion, aspects of the present invention provide an assembled, optimized, and evaluated a revolutionary depth-sensitive MTR-LSCI technology for continuous, fast, and high-resolution imaging of cerebral hemodynamics. The performance of MTR-LSCI was evaluated through experiments conducted on head-simulating phantoms and in-vivo studies in adult rodents. These pilot studies demonstrated that MTR-LSCI enabled mapping CBF variations at different depths with a sampling rate of up to 1 Hz and varied spatial resolutions from tens of micrometers on tissue surface to 1-2 millimeters in the deep brain. The results are generally in agreement with previous studies utilizing other cerebral monitoring techniques and similar experimental protocols.

Incorporation of advanced SPAD cameras with improved gate widths and fill factors (via integrating microlenses) holds promise for enhancing the performance of MTR-LSCI. Additionally, integration of multiple NIR wavelengths and higher sampling rates would enable simultaneous imaging of multiple tissue hemodynamic parameters and the exploration of brain functional connectivity. With further improvement and validation in larger populations against established methods, a noninvasive, noncontact, fast, high-resolution, portable and affordable brain imager can be provided for fundamental neuroscience research in animals and translational studies involving human subjects. Moreover, MTR-LSCI holds the potential to be used for noninvasive assessment and therapeutic monitoring of many vascular and cellular diseases associated with abnormal tissue hemodynamics including cerebral disease, cardiovascular disease, peripheral vascular disease, cancer, diabetes, burn/wound injury, angiogenesis or tissue/vascular reconstruction injury.

### Summary

All the features disclosed in this specification, including any accompanying abstract and drawings, may be replaced by alternative features serving the same, equivalent or similar purpose, unless expressly stated otherwise. Thus, unless expressly stated otherwise, each feature disclosed is one example only of a generic series of equivalent or similar features.

Claim elements and steps herein may have been numbered and/or lettered solely as an aid in readability and understanding. Any such numbering and lettering in itself is not intended to and should not be taken to indicate the ordering of elements and/or steps in the claims.

Many alterations and modifications may be made by those having ordinary skill in the art without departing from the spirit and scope of the invention. Therefore, it must be understood that the illustrated embodiments have been set forth only for the purposes of examples and that they should not be taken as limiting the invention as defined by the following claims. For example, notwithstanding the fact that the elements of a claim are set forth below in a certain combination, it must be expressly understood that the invention includes other combinations of fewer, more or different ones of the disclosed elements.

The words used in this specification to describe the invention and its various embodiments are to be understood not only in the sense of their commonly defined meanings, but to include by special definition in this specification the generic structure, material or acts of which they represent a single species.

The definitions of the words or elements of the following claims are, therefore, defined in this specification to not only include the combination of elements which are literally set forth. In this sense it is therefore contemplated that an equivalent substitution of two or more elements may be made for any one of the elements in the claims below or that a single element may be substituted for two or more elements in a claim. Although elements may be described above as acting in certain combinations and even initially claimed as such, it is to be expressly understood that one or more elements from a claimed combination can in some cases be excised from the combination and that the claimed combination may be directed to a subcombination or variation of a subcombination.

Insubstantial changes from the claimed subject matter as viewed by a person with ordinary skill in the art, now known or later devised, are expressly contemplated as being equivalently within the scope of the claims. Therefore, obvious substitutions now or later known to one with ordinary skill in the art are defined to be within the scope of the defined elements.

The claims are thus to be understood to include what is specifically illustrated and described above, what is conceptually equivalent, what can be obviously substituted and also what incorporates the essential idea of the invention.

What is claimed is:

1. A system for noncontact, multiwavelength, time-resolved laser speckle contrast imaging (MTR-LSCI) of tissue blood flow, tissue blood oxygenation, and metabolic rate of tissue oxygen consumption in a subject, comprising:
at least two pulsed laser sources, each capable of emitting light pulses in nanosecond or picosecond width at near-infrared (NIR) range of 600-1100 nm, for illuminating tissue;
at least one diffuser in front of each of the at least two pulsed laser sources to generate a wide-field illumination;
a time-gated camera;
a controller to synchronize the time-gated camera and the at least two pulsed laser sources at 10-80 MHz for data collection;
a computing device having a processor for processing data to generate hemodynamic images on a display.

2. The system of claim 1, further comprising algorithms incorporating parallel computation and convolution functions to process received images and generate the hemodynamic images to the display.

3. The system of claim 1, wherein the time-gated camera has a gate step resolution of picoseconds.

4. The system of claim 1, wherein the time-gated camera has a gate width of nanoseconds.

5. The system of claim 1, wherein the time-gated camera has a spatial resolution of at least 256×512 single-photon-counting pixels.

6. The system of claim 1, further comprising at least one filter within the time-gated camera path to minimize an impact of ambient light on a detection NIR spectra.

7. The system of claim 1, further comprising at least two polarizers across each of the at least two pulsed laser sources and the time-gated camera path to reduce an influence of source reflections directly from a tissue surface.

8. The system of claim 1, further comprising at least one zoom lens attached to the time-gated camera to adjust the region-of-interest (ROI)/field-of-view (FOV).

9. The system of claim 1, wherein the subject is one of a human or an animal.

10. A method of measuring tissue blood flow, tissue blood oxygenation, and metabolic rate of tissue oxygen consumption at different depths in a subject, comprising:
positioning a multi-wavelength, time-resolved laser speckle contrast imaging (MTR-LSCI) device in proximity to the subject, the MTR-LSCI device including:
at least two pulsed laser sources, each capable of emitting light pulses in nanosecond or picosecond width at near-infrared (NIR) range of 600-1100 nm, for illuminating tissue;
at least one diffuser in front of each of the at least two pulsed laser sources to generate a wide-field illumination;
at least one optical switch to switch between the at least two pulsed light sources;
a time-gated camera;
at least one zoom lens attached to the time-gated camera to adjust the ROI/FOV;
a controller to synchronize the time-gated camera and the at least two pulsed laser sources at 10-80 MHz for data collection; and
a computing device having a processor for processing data to generate hemodynamic images to a display;
using the at least two pulsed laser sources to apply pulsed widefield illumination at multiple wavelengths;
setting the at least two pulsed laser sources to different wavelengths; and
measuring at least one of tissue blood flow, tissue blood oxygenation, and metabolic rate of tissue oxygen consumption in the subject.

11. The method of claim 10, wherein the method maps blood flow (BF) distributions at different depths of target tissue, wherein the imaging depth depends on light propagation inside a tissue volume, captured by the time-gated camera.

12. The method of claim 10, wherein the method maps blood flow (BF) distributions at different depths of target tissue, wherein the BF is calculated based on detected diffuse laser speckle fluctuations resulting from motions of red blood cells in a target tissue volume.

13. The method of claim 10, wherein the method maps parameters of oxy-hemoglobin concentration ([HbO₂]), deoxy-hemoglobin concentration (MN), and tissue blood oxygen saturation (StO₂) at different depths of target tissue, wherein the parameters are calculated based on detected light intensity attenuations by a target tissue volume.

14. The method of claim 10, wherein the method determines a TMRO₂, wherein the TMRO₂ is calculated based on a measured BF and StO₂.

15. The method of claim 10, wherein the tissue is any part of a body of the subject.

16. The method of claim 10, wherein the subject is either healthy or is diagnosed with a disease associated with altered tissue blood flow and oxygenation, cerebral disease, cardiovascular disease, peripheral vascular disease, cancer, diabetes, burn/wound injury, angiogenesis or tissue/vascular reconstruction injury.

17. The method of claim 10, wherein the subject is under medical interventions.

18. The method of claim 10, further comprising determining an efficacy of interventions.

19. An integrated instrument for performing MTR-LSCI measurements, comprising:
at least two nanosecond or picosecond pulsed NIR lasers, coupled with diffusers to deliver wide-field illumination to a target tissue volume;
a time-gated, single-photon avalanche diode (SPAD) camera to capture intensity images and deliver the intensity images to a computer processor, wherein the computer processor includes control software to synchronize the at least two pulsed lasers and the time-gated, SPAD camera for data collection;
a computing device to process received images using parallel computation and convolution functions and generate hemodynamic images on a display.

20. The instrument of claim 19 wherein the MTR-LSCI instrument is in proximity to, but not directly touching, the subject.

21. The instrument of claim 19, wherein a microlens array is attached to the camera to enhance the detection sensitivity.

22. The instrument of claim 19, wherein wavelengths of the at least two pulsed lasers are set to 785 nm and 830 nm.
